(19) United States
(12) Patent Application Publication
(10) Pub. No.: US 2005/0169987 A1
(43) Pub. Date: Aug. 4, 2005

(54) Compacted menthol

(76) Inventor: Alfred Korber, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/516,005

(22) PCT Filed: May 24, 2003
(86) PCT No.: PCT/EP03/05452

(30) Foreign Application Priority Data
May 31, 2002 (DE) 102 24 087.6

(51) Int. Cl. A61K 31/045; A61K 9/20

(52) U.S. Cl. 424/464; 514/729

(57) ABSTRACT

The invention provides menthol mouldings, a process for producing menthol mouldings and the use thereof.

The invention provides menthol mouldings, a process for producing menthol mouldings and the use thereof.

L-menthol has a unique, refreshing taste, a minty odour and a pronounced cooling effect on the skin and mucous membranes. It is used, for example, in oral hygiene, in cosmetic and pharmaceutical preparations, in tobacco and in confectionery, as described in e.g. Perfumer&Flavorist, vol. 13, October-November 1988, p. 37.

L-menthol is the main constituent of peppermint oils from Mentha arvensis (concentration: 70 to 80%) and Mentha piperita (concentration: 50 to 60%). L-menthol is obtained from crude peppermint oil by crystallisation. Depending on the method of crystallisation and the starting material, the crystals differ in taste and in crystal size and shape (Perfumer&Flavorist, vol. 22, November-December 1997, p. 1).

Many processes for preparing synthetic menthol have been disclosed. One economically viable preparation of synthetic l-menthol starts, for example, from thymol. L-menthol is obtained from the four stereoisomeric menthols formed by hydrogenation, via several process steps, with a chemical purity of >99% and an enantiomer purity of >99% (e.g. in Bauer, Garbe, Surburg, Common Fragrance and Flavor Materials, 4th ed., Wiley-VCH, Weinheim 2001, p. 52-55). The l-menthol resulting from this process is characterised sensorially by its purity and intensity.

L-menthol and racemic menthol are commercially available in many solid forms; conventional forms are, for example, powders, crystals, solidified distillates, flakes and mouldings. The menthol mouldings disclosed hitherto clump together after being stored for some time and this impairs their pouring and handling properties.

U.S. Pat. No. 3,064,311 describes l-menthol in a flaked form. In this case, distilled l-menthol is melted and a thin molten film layer is applied to a supercooled surface. The solidified l-menthol film is broken into small pieces. The product of this process is a brittle l-methol flake which has a thickness of 0.125 to 1.25 mm and a size of 3 to 25 mm. The flaked l-menthol prepared in this way, as described in the relevant document, becomes sticky and the flakes clump together after 24 hours.

However, mouldings of l-menthol have been disclosed which may exhibit caking or clumping together after some time, depending on the time and conditions of storage. These l-menthol mouldings are produced from flaked l-menthol or from mixtures of flaked l-menthol and crystalline l-menthol, herein the total concentration of alpha-l-menthol is less than 50 wt. %.

The object of the present invention is, therefore, the provision of menthol mouldings which ensure high storage stability, simple handling characteristics and good pouring properties.

Therefore the present invention provides menthol mouldings which are characterised in that the concentration of alpha-menthol in the moulding is greater than or equal to 70 wt. %.

The present invention also provides a process for producing menthol mouldings, characterised in that the menthol used to produce the mouldings has a concentration of alpha-menthol of at least 70 wt. %.

The invention also provides the use of these mouldings for incorporation into products, specifically for the flavouring of products, in particular foodstuffs, tobacco products, scented mixtures, oral hygiene products and cosmetic, pharmaceutical and dermatological products.

Alpha-menthol is known per se and is described in detail in, for example, J. Am. Chem. Soc. 1917, p. 1515 or also in J. Chem. Soc. 101, p. 118, and has a melting point of 42 to 43° C.

The preparation of alpha-menthol has also been disclosed (e.g. in DE-A 2109456 or DE-A 2530481). In addition, l-menthol crystallised by this process is commercially available in the form of white crystals (Haarmann & Reimer, Holzminden).

The concentration of alpha-menthol in the moulding is preferably greater than or equal to 80 wt. %, particularly preferably greater than or equal to 90 wt. % and very particularly preferably greater than or equal to 95 wt. %. In a preferred embodiment, the concentration of alpha-menthol in the moulding is greater than 98 wt. %.

The mouldings may be produced from d-menthol, l-menthol and any mixture thereof; l-menthol, d-menthol and racemic menthol are preferred.

The alpha-menthol used in the moulding may be of synthetic or natural origin; synthetic material is generally used. Obviously, any mixture of synthetic and natural menthol as well as of racemic and enantiomerically pure menthol may also be used.

The alpha-menthol used to produce the mouldings may be present in any form, crystals being preferred. These crystals preferably have crystal sizes in the range 50-500 μm, particularly preferably in the range 100 to 300 μm and very particularly preferably in the range 150 to 250 μm.

The proportion of crystalline alpha-menthol is preferably at least 80 wt. %, particularly preferably at least 90 wt. % and particularly preferably at least 95 wt. %, with respect to the entire amount of alpha-menthol in the moulding. In a very particularly preferred embodiment, all of the alpha-menthol used is in the form of crystalline alpha-menthol.

The mouldings may be present in the form of, for example, spheres, cubes, cuboids, cushions, cylinders, tablets, pellets or briquettes, a preferred form being pellets.

The shape of mouldings according to the invention formed by compression (compaction) remains stable when stored for several months. Clumping together, caking or coalescence of the mouldings does not occur. Convenient and safe handling as well as the capacity to pour, flow freely and meter out the mouldings according to the invention is therefore provided.

Compression of the menthol may be performed in a wide variety of ways. Proven methods for making briquettes are described, for example, in Chemie−Anlagen+Verfahren 1985, vol. 4, pages 51, 54 and 59.

For compaction purposes, the menthol is generally converted into a moulding in a compression process. The dimensions of the moulding may vary greatly. In the case of a sphere, the diameter is in the range 1 mm to 50 mm, preferably 5 to 20 mm. In the case of pellets, the length and width are in the range 3 to 50 mm, preferably 5 to 15 mm. The depth is in the range 0.5 to 20 mm, preferably 2 to 8 mm.

During compaction, a moulding pressure in the range 5 to 200 kN (kiloNewtons) is generally applied in the compacting machine. The moulding pressure is preferably 10 to 100 kN, particularly preferably 20 to 80 kN.

The following example explains the invention in more detail.

### EXAMPLE

150 to 200 kg of synthetic l-menthol crystals (concentration of alpha-menthol >98.5 wt. %) are supplied hourly, via a vibrating chute, to a compacting machine (from Bepex GmbH) consisting of two compacting rollers (circumference 60 cm, width 10 cm, area 600 cm²). At room temperature and with a moulding pressure of 50 kN, the compactor produces mouldings in the form of pellets (cushion-shaped) with the external dimensions length=width=10 mm and depth=6 mm. The mouldings produced in this way exhibit no clumping together or caking, even after storage for three months at room temperature.

1. Menthol mouldings comprising alpha-menthol, wherein the concentration of alpha-menthol in the moulding is greater than or equal to 70 wt. %.

2. Mouldings according to claim 1, characterized in that the concentration of alpha-menthol in the moulding is greater than or equal to 80 wt. %.

3. Mouldings according to claim 1, wherein the menthol is l-menthol.

4. Mouldings according to claim 1, wherein the proportion of crystalline alpha-menthol is at least 80 wt. %, with respect to the entire amount of alpha-menthol in the moulding.

5. Mouldings according to claim 4, wherein the proportion of crystalline alpha-menthol is at least 90 wt. %, with respect to the entire amount of alpha-menthol in the moulding.

6. Mouldings according to claim 5, wherein all of the alpha-menthol used is in the form of crystalline alpha-menthol.

7. Mouldings according to claim 1 in the from of spheres, cubes, cuboids, cushions, cylinders, tablets, pellets or briquettes.

8. A process for producing menthol mouldings, wherein the menthol used to produce the mouldings has a concentration of alpha-menthol of at least 70 wt. %.

9. A process according to claim 8, wherein the menthol used to produce the mouldings has a concentration of alpha-menthol of at least 80 wt. %.

10. A process according to claim 8, wherein the proportion of crystalline alpha-menthol is at least 80 wt. %, with respect to the entire amount of alpha-menthol in the moulding.

11. A process according to claim 10, wherein in that the proportion of crystalline alpha-menthol is at least 90 wt. %, with respect to the entire amount of alpha-menthol in the moulding.

12. (canceled)

13. A tobacco product comprising the moulded menthol of claim 1.

14. A process for moulding menthol according to claim 8, wherein said menthol is formed into a moulded shape by compaction.

15. A process for moulding menthol according to claim 14, wherein a compaction moulding pressure is applied within a range from 10 to 100 kN.
